(19) United States
(12) Patent Application Publication
(10) Pub. No.: US 2018/0085060 A1
(43) Pub. Date: Mar. 29, 2018

(54) BRAIDED ELASTIC CONDUCTIVE STRIPE AND METHODS OF UTILIZING THEREOF

(71) Applicant: HEALTHWATCH LTD., Herzliya (IL)

(72) Inventors: Boaz SHOSHANI, Raanana (IL); Uri AMIR, Or Yehuda (IL)

(73) Assignee: HEALTHWATCH LTD., Herzliya (IL)

(21) Appl. No.: 15/809,929

(22) Filed: Nov. 10, 2017

Related U.S. Application Data
(63) Continuation-in-part of application No. 15/121,334, filed on Aug. 24, 2016, now abandoned.
(60) Provisional application No. 62/006,102, filed on May 31, 2014.
(60) Provisional application No. 61/950,139, filed on Mar. 9, 2014.

(51) Int. Cl. A61B 5/00; A61B 5/0408

(52) U.S. Cl. CPC A61B 5/6805; A61B 5/04085

(57) ABSTRACT

According to the teachings of the present invention there is provided an elastic smart garment. The garment includes an elastic tubular form having variable elasticity and at least one conductive textile electrode, for sensing electrical vital signals, such as a clinical level ECG signal. The garment further includes at least one elastic and loose conductive stripe, having a first end and a second end. The first end of the at least one conductive stripe is securely attached to a respective conductive textile electrode, and the second end of the at least one conductive stripe is operatively connected with a processor. The elasticity and looseness of the at least one conductive stripe is configured to prevent a pulling force from being applied to the respective conductive textile electrode, when the garment is stretched. When a conductive stripe is stretched by up to 15%, its resistance increases by less than 25%.

### CROSS REFERENCE TO RELATED APPLICATIONS

This application is a continuation in part of U.S. application Ser. No. 15/121,334 filed Aug. 24, 2016, which was a 371 National Stage entry of International Application Serial No. PCT/IL2015/050239 filed Mar. 5, 2015, and further claims the benefit under 35 USC 119(e) from U.S. provisional application 62/006,102 filed May 31, 2014, and the benefit under 35 USC 119(e) from U.S. provisional application 61/950,139 filed Mar. 9, 2014. The contents of each of these applications are hereby incorporated herein by reference in their entirety as if set forth verbatim.

This application also relates to the PCT/IL2013/050963, the disclosure of which is included herein by reference in its entirety

### FIELD OF THE INVENTION

The present invention relates to real-time health monitoring systems and more particularly, the present invention relates to a knitted garment having an elastic tubular form at preconfigured locations, transferring physiological signals such as 12-lead clinical level ECG or other signals from textile electrodes to a selected area of the garment.

### BACKGROUND OF THE INVENTION AND PRIOR ART

Monitoring systems for monitoring of physiological parameters of a living being are well known in prior art. For example, PCT/IL2012/000248, the disclosure of which is included herein by reference in its entirety, discloses a wearable health monitoring system that continuously checks the wellbeing of a person that, typically, is considered healthy, covering a significant range of health hazards that may cause a significant life style change/limitation, and provides an alert as early as possible—all this, with no significant limitation to the normal life style of the person bearing the system.

Unlike conventional gel electrodes, which are directly applied to the living being's skin, using a conductive gel, textile electrodes are dry contact sensors adapted for use in measuring ECG signals and other vital signals such (EEG), electroencephalogram (EOG), electrooculogram and other medical measurements on the skin without any skin preparation, such as needed with wet electrodes, for example, shaving hairy skin.

To improve performance over conventional wet ECG sensors and to be able to conduct continuous long term monitoring, a textile substrate is used to develop dry textile electrodes for sensing physiological parameters of a living being such as ECG signals. One such textile electrodes are disclosed in PCT application PCT/IL2013/050964, filed Nov. 23, 2013, titled “float loop textile electrodes and methods of knitting thereof”, the disclosures of which is included herein by reference for all purposes as if fully set forth herein.

There is however a need to transfer the sensed electrical signals from the textile electrodes to a processing unit for collecting and processing the sensed data.

Reference is made to FIG. 1 (prior art) depicting an open smart garment 20, having multiple textile electrodes 50 integrally knitted therein. Smart garment 20 is configured to receive a processing unit 70. FIG. 1 demonstrates the need to electrically connect each of the textile electrodes 50 to processing unit 70.

One solution is to integrally knit conductive traces form each of the textile electrodes 50 to a docking station configured to receive processing unit 70. This solution is disclosed in PCT application PCT/IL2013/050963, titled “vertical conductive textile traces and methods of knitting thereof”, filed Nov. 23, 2013, the disclosures of which is included herein by reference for all purposes as if fully set forth herein.

FIG. 2a (prior art) schematically illustrates an example garment 20, having an elastic tubular form, typically, with no limitations, a knitted tubular form, wherein textile electrodes 50 are knitted therein and are individually operatively connected to a processing unit 70. FIG. 2b (prior art) depicts a front view of an example garment, wherein the textile electrodes 50 are designed to measure at least 12-lead ECG signal, and are connected to a processing unit (not shown) by respective conductive traces 60, knitted therein.

The conductive traces 60 are knitted therein as part of the fabrication of the garment, wherein the conductivity, in particular between adjacent knitting courses in the vertical direction, can support the transfer of clinical level ECG signals from a textile electrode, along the fabric, to a selected area in the garment preconfigured to host the processing unit. Since the normal knitting direction of a tubular form is substantially horizontal, conductive traces 90 that are knitted therein in a horizontal direction maintain a stable conductivity.

The good conductivity should prevail when the fabric is stretched to different directions during wearing, which typically requires that the conductive physical means for transferring the sensed electrical signals from textile electrodes 50 to processing unit 70. This may entail that the conductive physical means is made of materials having high elasticity. This may entail that good conductive should prevail when the fabric is stretching, in particular between adjacent knitting courses in the vertical direction. However, naturally, when the integrally knitted conductive traces 60 are stretched, tight as it is, gaps of air are formed between the knitted loops, thereby reducing the conductivity of the integrally knitted conductive traces 60.

The good conductivity of the conductive physical means should prevail when using any type of basic fabric yarns (cotton, synthetic yarns, metallic yarns, etc.).

The good conductivity should prevail after a preconfigured number of washes, including in a washing machine.

The good conductivity should prevail in any knitting design, location and shape in the fabric.

More so, signals detecting is the motion artifact occurring during movement of the person 10, wearing garment 20. The motion artifact problem may increase as a result of the large area of the textile electrodes 50 and/or the conductive traces 60, moving with respect to the skin of user 10. It should be noted that the larger the area of the textile electrodes 50 and/or the conductive traces 60 is, the higher the capacitance between the skin and textile electrode 50 and conductive traces 60 is.

There is therefore a need and it would be advantageous to provide conductive physical means for transferring the sensed electrical signals from textile electrodes to a target receiving unit that provides high conductivity and low sensitivity to motion artifacts, wherein the textile electrodes are an integral part of a seamless garment, having a tubular form.

US patent application 2010/0185076, by Jeong et al, “Jeong” discloses a physiological signal measurement garment and a physiological signal processing system. The physiological signal measurement garment includes: a main garment body which is formed of an elastic fabric and includes a mesh structure and an elastic band; at least one physiological signal sensing electrode sewn on the garment body; a physiological signal transmission unit which is sewn on the garment body and transmits a physiological signal sensed by the physiological signal sensing electrode; and a physiological information measurement module that measures various kinds of physiological information from the physiological signal, which is sensed by the physiological signal sensing electrode and transmitted through the physiological signal transmission unit.

However, the garment is not a seamless tubular form, and it is tailored using elastic seams formed in the garment by considering the muscular shapes, and an elastic mesh structure is inserted for a buffering action so that the muscular and skin motion can be sufficiently absorbed. In particular, on the front of the garment, elastic seams are formed to correspond to the boundaries of the trapezius muscle, pectoralis major muscle, rectus abdominis muscle, and external oblique abdominal muscle, and an elastic mesh is inserted. In addition, considering the motion of upper arms, an elastic band is inserted into a part over the serratus anterior muscle and the external oblique abdominal muscle. It should be noted that when talking about the need of considering the motion of upper arms, one must note that, in his disclosure, Jeong does not relate, in any way, to the LA/RA electrodes that are critical in measuring clinical level ECG. It should be further noted that it makes no economical-sense to tailor a garment for each person according to his/her muscle structure.

In addition, in order to minimize the motion of the pectoralis major muscle as the upper arms move, Jeong suggests a buffering elastic mesh structure is inserted into an elastic seam near the armpit. On the rear of the garment, according to the same principle as applied to the front of the garment, elastic seams are formed to correspond to the boundaries of the teres major muscle, trapezius muscle, and teres minor muscle, and an elastic mesh structure is inserted.

This design of the garment dictates different electrodes than the integrated textile electrodes of the present invention, the integrated textile electrodes being part of a seamless garment, having a tubular form. This dictates substantially different signal quality, and still the presented 4-electrodes design and placement suggest the inability to provide clinical level ECG signals, certainly not a 12-lead clinical level ECG, as does the present invention.

### Definitions

The term “seamless monitoring”, as used herein with conjunction with wearable monitoring devices, refers to a device that when worn by an average person, wherein the device puts no significant limitation to the normal life style of that person and preferably not seen by anybody when used and not disturbingly felt by the user while wearing it. Furthermore, no activity is required from the monitored person in order for the system to provide a personal-alert when needed. It should be noted that people that pursue non-common life style, such as soldiers in combat zone or in combat training zone, or firefighters in training and action, or athletes in training or competition may utilize non-seamless monitoring devices. As the “seamless monitoring” characteristics refers also to the user's behavior, the wearable component is preferably an item that is normally worn (e.g., underwear) and not some additional item to be worn just for getting the alert. It should be noted that the term “seamless monitoring” differ from the notion of commonly known notion of a seamless clothing item that refers to tubular form clothing having no seams for forming the tubular form.

The term “garment”, as used herein with conjunction with wearable clothing items, refers to wearable clothing items with seamless monitoring capabilities that preferably, can be tightly worn adjacently to the body of a monitored living being, typically adjacently to the skin, including undershirts, sport shirts, brassiere, underpants, special hospital shirt, socks and the like. Typically, the term “garment”, as used herein, refers to a clothing item that is worn adjacently to the external surface of the user's body, under external clothing or as the only clothing, in such way that the fact that there are sensors embedded therein, is not seen by any other person in regular daily behavior.

The terms “course” and “line segment”, are used herein as related terms. The tubular form of the garment is knitted on a knitting machine, such as a Santoni knitting machine, where the tubular form is knitted in a spiral having substantially horizontal lines. A single spiral loop/circle us referred to herein as a course and a portion of a course is referred to as line segment.

The term “vertical conductive trace”, is used herein, refers to knitting a lead wire, made of conductive yarns, and capable of transferring electrical signals across knitted line segment.

The phrase “clinical level ECG”, as used herein with conjunction with ECG measurements, refers to the professionally acceptable number of leads, sensitivity and specificity needed for a definite conclusion by most cardiology physicians to suspect a risky cardiac problem (for example, arrhythmia, myocardial ischemia, heart failure) that require immediate further investigation or intervention. Currently, it is at least a 12-leads ECG and preferably 15-lead ECG, coupled with a motion/posture compensation element, and a real-time processor with adequate algorithms.

### BRIEF SUMMARY OF THE INVENTION

A principle intention of the present invention is to provide conductive physical means for transferring the sensed electrical signals from textile electrodes to a target receiving unit. Typically, the conductive physical means is composed of groups of elastic conductive yarns to thereby form conductive stripes or conductive ribbons, herein referred to as a “conductive stripe”. The conductive stripe is made of yarns selected form a group of yarns including synthetic yarns and metallic yarns. The conductive stripe provides high conductivity, elasticity and low sensitivity to motion artifacts.

The conductive stripes maybe in the form of rounded stripes, flat stripes or any other cross-sectional shape. One way to achieve high conductivity, is to increase the number of conductive yarns in the conductive stripe. However, when using rounded conductive stripe, the stripes tend to become bulky. Another way to achieve high conductivity, is to construct the conductive stripes from groups of yarns using braiding technology. The braiding technology enhance the contact between conductive yarns, in particular, while in stretching conditions. The flat conductive stripe is also more convenient logistically, when using rolls of conductive stripes.

Another principle intention of the present invention is to connect textile electrodes to a signal receiving unit by a flexible and loose conductive stripe, such that the conductive stripe does not apply pulling forces or applies minimal pulling forces on the textile electrode securely connected thereto. Thereby, during motion, the textile electrode remains stably in position with respect to the skin of the user, while the signals, such as ECG signals, transfer to a receiving unit such as a docking station.

It should be noted that the signals can be any sensed electric signals (e.g. respiration) and it is not restricted to ECG signals. It should also be noted that any non-horizontal angle can be knitted using this invention by a continuous sequence of vertical lines.

It should be further noted that with respect to the embodiments provided by PCT application PCT/IL2013/050963, the embodiments of the present invention show significant reduction of motion artifact when the user is in motion, due to the fact that the new conductive elastic stripes are attached to the basic garment only in a few points such as to prevents the pulling the respective electrodes, which pulling may create unnecessary friction of the textile electrode with the skin. Furthermore, the present invention provides embodiment that substantially reduce the quantity and cost of materials and labor.

It should be further noted that the present invention will be often described in terms of the smart garment being knitted, in order to provide the required variable elasticity. However, the garment is not limited to being a knitted smart garment, and may be manufactured using other technologies, such as polymer based garments that are produced using 3D printing technologies.

According to the teachings of the present invention there is provided an elastic smart garment, such as a knitted smart garment. The garment includes an elastic tubular form having a preconfigured elasticity, typically variable elasticity, and at least one conductive textile electrode for sensing an electrical vital signal, such as a clinical-level ECG signal wherein the conductive textile electrode is integrally manufactured with the tubular form. The elastic tubular form includes a skin side and an external side, wherein the external side faces away from the user's skin.

Typically, with no limitations, the elastic tubular form is a knitted tubular form. The invention may be described, hereon, in terms of the elastic tubular form being a knitted tubular form. However, the elastic tubular form being, typically, a knitted tubular form, not limited to be a knitted tubular form, and the elasticity may be obtained by other means.

The garment further includes at least one elastic conductive stripe, having a first end and a second end.

The first end of the at least one conductive stripe is securely and conductively attached to a respective conductive textile electrode, and the second end of the at least one conductive stripe is operatively connected with a processor, facilitating the sensed vital signal to be communicated from the least one conductive textile electrode to the processor. The second end of the at least one conductive stripe may be securely attached to a connector, such as, with no limitations, a HDMI connector. Alternatively, the second end of the second end of the at least one conductive stripe is securely attached to a docking station.

The elasticity of the at least one conductive stripe is configured to prevent a pulling force from being applied to the respective conductive textile electrode, when the garment is stretched.

The at least one conductive stripe is insulated by insulation means, wherein the insulation means are selected from the group including at least one insulating adhered stripe, sleeves, non-conductive coating and non-conductive textile material that is knitted, weaved, braided or covered on the respective at least one conductive stripe.

Optionally, at least a portion of the yarns, from which the at least one conductive stripe is composed of, are braided.

Preferably, the yarns, from which the at least one conductive stripe is composed of, are braided.

Optionally, sideways movements of the at least one conductive stripe is restricted by a motion restricting means, wherein the motion restricting means is securely attached to the garment, and wherein the at least one conductive stripe is free to move within the restricted space as provided by the motion restricting means.

Optionally, the motion restricting means is selected from the group of motion restricting means group consisting of a sleeve, sewn-in yarns that are sewn over the at least one conductive stripe, and a combination thereof.

Preferably, the motion restricting means is securely attached to the skin side of the garment.

The insulation means are designed not reduce the conductivity of the respective the at least one conductive stripe. The insulation means are further designed not reduce the local elasticity of the respective the at least one conductive stripe.

The insulation means is configured to prevent the at least one conductive stripe conductive stripe from being electrically shortened by any one of the user's skin, a neighboring conductive stripe or a neighboring textile electrode.

Typically, the at least one conductive stripe is at least partially loose inside the respective insulation means.

The at least one conductive stripe is made of yarns selected form a group of yarns including synthetic yarns and metallic yarns, or a combination thereof.

When a conductive stripe is stretched in length by up to 15%, the electric resistance of the conductive stripe increases by less than 25%, with respect to the non-stretched, rest state of the conductive stripe. When the conductive stripe is further stretched in length beyond the 15% and up to 30% of the rest state sate length, the electric resistance increases by less than 10%, with respect to the non-stretched, rest state of the conductive stripe. When the conductive stripe is further stretched in length beyond the 30% of the rest state sate length, the electric resistance increases by less than 5%, with respect to the non-stretched, rest state of the conductive stripe.

The garment may include a zipper, wherein the zipper is situated between the at least one textile electrode and a docking station, wherein the at least one conductive stripe passes through the continuous section of the garment, without crossing the zipper, and wherein the second end of the respective at least one conductive stripe or knitted line-trace is securely attached to the docking station.

### BRIEF DESCRIPTION OF THE DRAWINGS

The present invention will become fully understood from the detailed description given herein below and the accompanying drawings, which are given by way of illustration and example only and thus not limitative of the present invention, and wherein:

FIG. 1 (prior art) depicts an open smart garment, having multiple textile electrodes integrally knitted therein, wherein the smart garment is configured to receive a processing unit.

FIG. 2a (prior art) is a schematic illustration of an example garment, having a tubular form, wherein textile electrodes are knitted therein.

FIG. 2b (prior art) depicts a front view of an example garment, wherein the textile electrodes are designed to measure a 15-lead ECG signal.

FIG. 3a depicts segments of a number of conductive stripes, according to embodiments of the present invention, wherein the conductive stripes are covered by an insulating tube, showing an open end of the conductive stripes.

FIG. 3b depicts segments of a number of conductive stripes, as in FIG. 3a, showing the other end of the conductive stripes, which, in the shown example, are connected to an HDMI connector.

FIG. 3c depicts an example segment of a rounded conductive stripe that is covered by an insulating tubular cover, according to some embodiments of the present invention.

FIG. 3d is a schematic illustration of a chart showing the relation of the overall resistance of a rounded conductive stripe and that of a flat conductive stripe wherein the resistance of the 1 meter of conductive stripes are shown as a function of stretching length of the respective conductive stripe.

FIG. 3e is a schematic illustration of a chart showing the average change in resistance, of flat conductive stripes of various lengths, with respect to the resistance of the respective conductive stripe at rest, after being stretched in segments of 10% of the length of the respective conductive stripe.

FIG. 3f is a schematic illustration of a chart showing the average change in resistance, of flat conductive stripes of various lengths, with respect to the with respect to the previous stretched state, after being stretched in segments of 10% of the length of the respective conductive stripe.

FIG. 4a depicts an example segment of a flat conductive stripe having a length of 16 cm (as measured by a ruler).

FIG. 4b depicts an example prior art conductive stripe having a length of 16 cm.

FIG. 5 illustrates an example smart garment, having multiple textile electrodes integrally knitted therein, wherein the conductive stripes are configured to transfer the sensed electrical signals from the textile electrodes to a processing unit configured to collect the sensed data, according to some embodiments of the present invention.

FIG. 6 illustrates an example method of securely connecting a conductive stripe to a respective textile electrode, according to some embodiments of the present invention.

FIGS. 7a and 7b illustrate example smart garments, having multiple textile electrodes connected to conductive stripes, wherein insulating sleeves are used to insulate the conductive stripes from being electrically shortened by an adjacent conductive stripe and/or the user's skin, according to some embodiments of the present invention.

FIGS. 7c and 7d depict another example garment, according to the methods shown in FIGS. 7a and 7b. FIG. 7c, illustrating the internal side of garment the garment, having multiple textile electrodes connected to respective conductive stripes.

FIG. 8 illustrates an example smart garment, having multiple textile electrodes connected to conductive stripes, wherein a lining is used to insulate the conductive stripes from being electrically shortened by the user's skin, according to some embodiments of the present invention.

FIG. 9 is a schematic illustration of an example garment having a tubular form and being an undershirt having a zipper in the front side, wherein textile electrodes are knitted therein.

FIG. 10 is a schematic illustration the example garment shown in FIG. 9, wherein the zipper is unzipped and the garment in a spread, unfolded form.

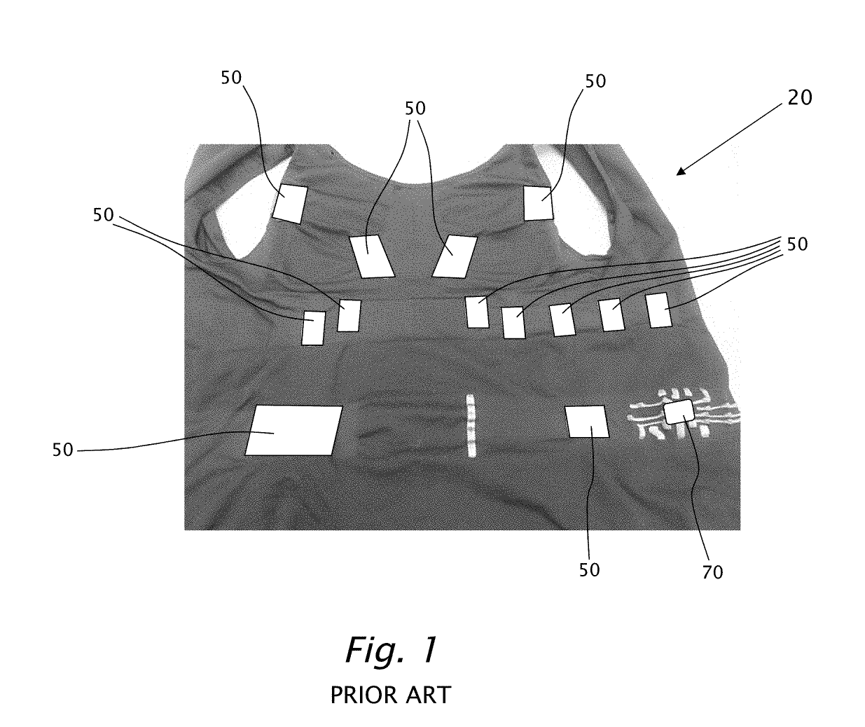

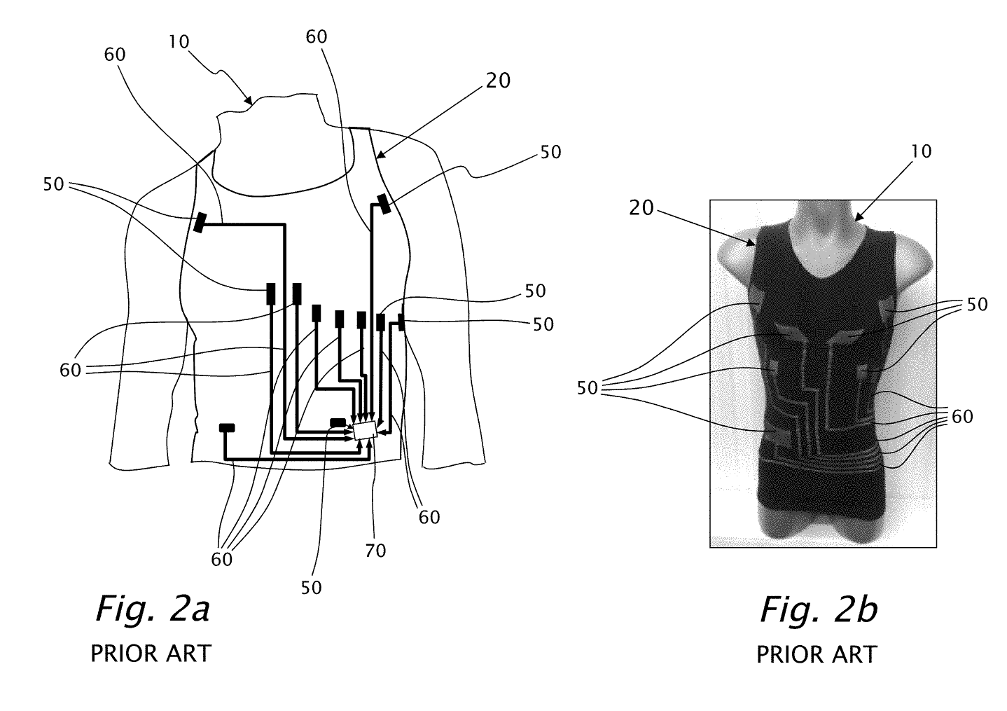

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein; rather, these embodiments are provided, so that this disclosure will be thorough and complete, and will fully convey the scope of the invention to those skilled in the art.

An embodiment is an example or implementation of the inventions. The various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention may also be implemented in a single embodiment.

Reference in the specification to “one embodiment”, “an embodiment”, “some embodiments”, “another embodiment” or “other embodiments” means that a particular feature, structure, or characteristic described in connection with the embodiments is included in at least one embodiments, but not necessarily all embodiments, of the inventions. It is understood that the phraseology and terminology employed herein is not to be construed as limiting and are for descriptive purpose only.

Methods of the present invention may be implemented by performing or completing manually, automatically, or a combination thereof, selected steps or tasks. The term “method” refers to manners, means, techniques and procedures for accomplishing a given task including, but not limited to, those manners, means, techniques and procedures either known to, or readily developed from known manners, means, techniques and procedures by practitioners of the art to which the invention belongs. The descriptions, examples, methods and materials presented in the claims and the specification are not to be construed as limiting but rather as illustrative only.

It should be noted that orientation related descriptions such as “bottom”, “up”, “horizontal”, “vertical”, “lower”, “top” and the like, assumes that the is worn by a person being in a standing position.

Meanings of technical and scientific terms used herein are to be commonly understood as to which the invention belongs, unless otherwise defined. The present invention can be implemented in the testing or practice with methods and materials equivalent or similar to those described herein.

A principle intention of the present invention is to connect textile electrodes to a signal receiving unit by an elastic and loose conductive stripe, such that the conductive stripe does not apply pulling forces or applies minimal pulling forces on the textile electrode securely connected thereto. Thereby, during motion, the textile electrode remains stably in position with respect to the skin of the user, while the signals, such as ECG signals, transfer to a receiving unit such as a docking station.

The conductive stripes maybe in the form of rounded stripes, flat stripes or of any other cross-sectional shape. One way to achieve high conductivity, is to increase the number of conductive yarns in the conductive stripe. However, when using rounded conductive stripes, the stripes tend to become bulky, and so is the garment as a whole. Another way to achieve high conductivity, is to construct the conductive stripes from groups of yarns using braiding technology. The braiding technology enhance the contact between conductive yarns, in particular, while in stretching conditions.

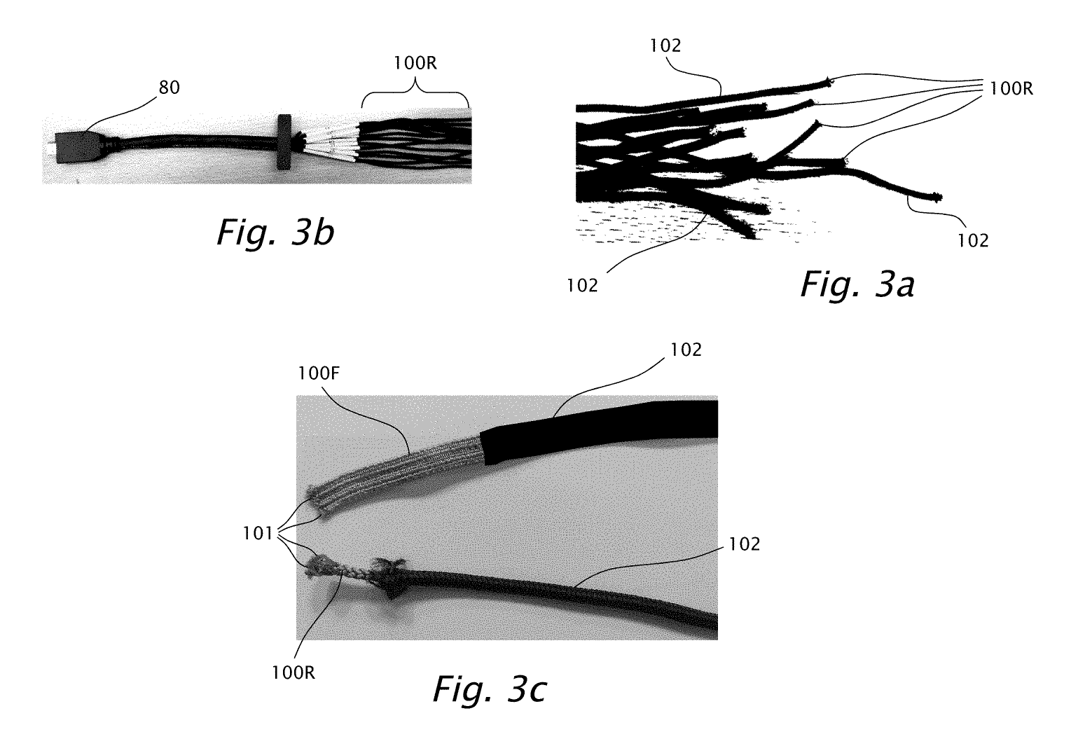

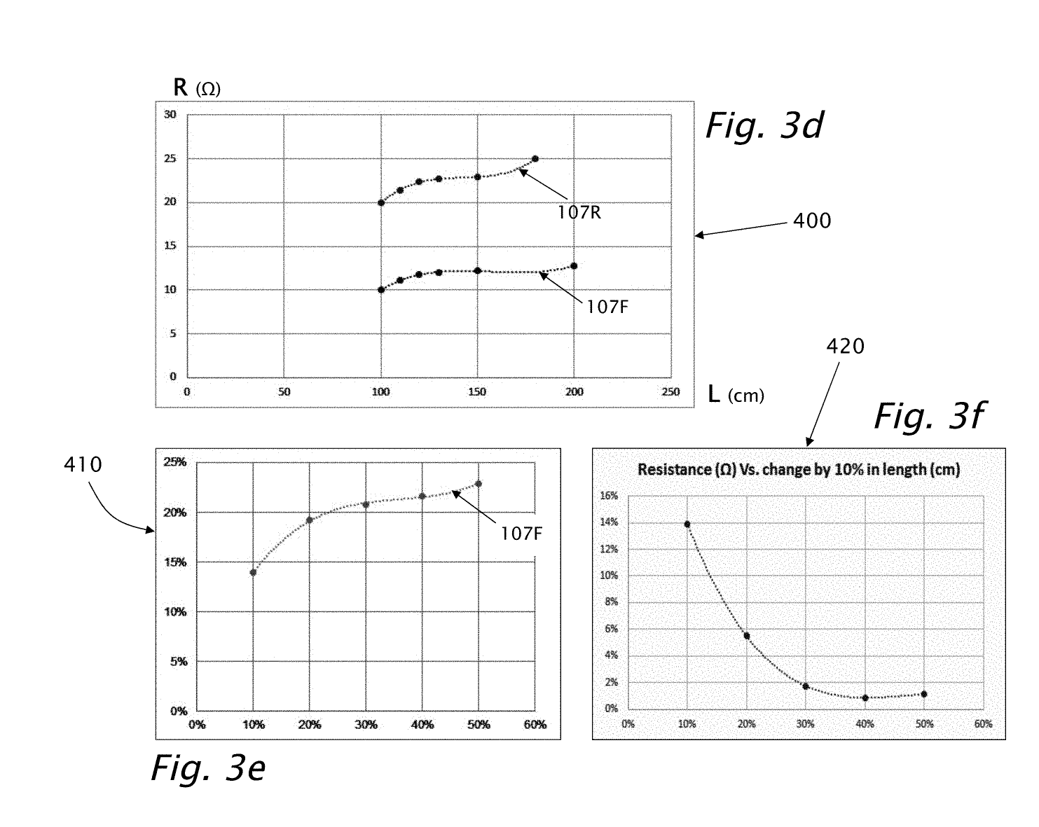

FIG. 3a depicts example segments of a number of rounded conductive stripes 100R that are covered by an insulating tubular cover 102, showing an open end of conductive stripes 100. FIG. 3b depicts example segments of a number of rounded conductive stripes 100, showing the other end of conductive stripes 100, which in the shown example, with no limitation, are connected to an HDMI connector 80. Insulating tubular cover 102 is elastic and does not limit the elasticity of conductive stripe 100. FIG. 3c depicts an example segment of a rounded conductive stripe 100R that is covered by an insulating tubular cover 102, and also an example segment of a flat conductive stripe 100F that is also covered by an insulating tubular cover 102.

Since, in this example, the flat conductive stripe 100F contains more conductive yarns 101, the overall resistance of rounded conductive stripe 100R is lower than that of flat conductive stripe 100F. The example chart 400 shown in FIG. 3d, shows the resistance (R) of conductive stripes (100R and 100F) as a function of stretching length (L), wherein the initial length of both stripes is 1 (one) meter. It can be observed that the base conductivity of flat conductive stripe 100F, having more conductive yarns 101, is 10 Ohms while the base conductivity rounded conductive stripe 100R is 20 Ohms. However, when stretching the stripes 100 by 10%, the resistance of both stripes 100 is growing, proportionately, by about 10% until the stripes 100 are stretched by about 20% in length, where the resistance starts to level off, as depicted in both graphs 107R and 107F. This is one of the major advantages of conductive stripes 100, which enables to maintain the good and stable conductivity and to obtain efficient ECG signals reading. It should be noted that conductive stripes 100 are predesigned to stretch no more than 10% and practically, conductive stripes 100 do not stretch more than 25%.

TABLE 1

| | Resistance (Ohm) vs. change in length (cm) | | | | | | |
|---|---|---|---|---|---|---|---|
| Stretched | Original | | | | | | |
| by | length | 0% | 10% | 20% | 30% | 40% | 50% |
| Stripe | 30 | 3.6 | 4 | 4.1 | 4.2 | 4.2 | 4.2 |
| length | 40 | 4.6 | 5.2 | 5.3 | 5.3 | 5.4 | 5.4 |
| | 50 | 5.4 | 6.2 | 6.6 | 6.7 | 6.6 | 6.7 |
| | 60 | 6.2 | 7.2 | 7.5 | 7.6 | 7.8 | 8.1 |
| | 70 | 7.2 | 8.3 | 8.8 | 8.8 | 8.9 | 9.2 |
| | 80 | 8 | 9.1 | 9.7 | 9.8 | 9.8 | 9.9 |
| | 90 | 9.2 | 10.3 | 10.7 | 11 | 11.1 | 11 |
| | 100 | 9.8 | 11.3 | 12 | 12.1 | 12.2 | 12.1 |

TABLE 2

| | | Original length (cm) | | | | | | | | |
|---|---|---|---|---|---|---|---|---|---|---|
| | | 30 | 40 | 50 | 60 | 70 | 80 | 90 | 100 | average |
| Stretched by (%) | 10% | 11% | 13% | 15% | 16% | 15% | 14% | 12% | 15% | 14% |
| | 20% | 14% | 15% | 22% | 21% | 22% | 21% | 16% | 22% | 19% |
| | 30% | 17% | 15% | 24% | 23% | 22% | 23% | 20% | 23% | 21% |
| | 40% | 17% | 17% | 22% | 26% | 24% | 23% | 21% | 24% | 22% |
| | 50% | 17% | 17% | 24% | 31% | 28% | 24% | 20% | 23% | 23% |

TABLE 3

| | | Resistance (Ohm) vs. change by 10% in length (cm) | | | | | | | | |
|---|---|---|---|---|---|---|---|---|---|---|
| | | Original length (cm) | | | | | | | | |
| | | 30 | 40 | 50 | 60 | 70 | 80 | 90 | 100 | average |
| Stretched by Δ10% | 10% | 11% | 13% | 15% | 16% | 15% | 14% | 12% | 15% | 14% |
| | 20% | 3% | 2% | 7% | 5% | 7% | 7% | 6% | 7% | 5.5% |
| | 30% | 3% | 0% | 2% | 2% | 0% | 2% | 4% | 1% | 1.8% |
| | 40% | 0% | 2% | −2% | 3% | 2% | 0% | 1% | 1% | 1% |
| | 50% | 0% | 0% | 2% | 5% | 4% | 1% | −1% | −2% | 1% |

With reference to Tables 1, 2 and 3, as well as to the charts (410 and 420, respectively) shown in FIG. 3e and FIG. 3f, another experiment was conducted with various lengths of flat braided conductive stripes 100F, to analyze the conductivity behavior of the flat conductive stripes 100F. FIG. 3e is a schematic illustration of chart 410 showing the average change in resistance, of flat conductive stripes 100F of various lengths, with respect to the resistance of the respective conductive stripe at rest, after being stretched in segments of 10% of the length of the respective conductive stripe. FIG. 3f is a schematic illustration of chart 420 showing the average change in resistance, of flat conductive stripes 100F of various lengths, with respect to the with respect to the previous stretched state, after being stretched in segments of 10% of the length of the respective conductive stripe. In all lengths of conductive stripes 100F, it has been observed that, while stretching the conductive stripes 100F by up to 20%, the resistance of the conductive stripes 100F increases, averagely, by 14%, with respect to the resistance when conductive stripes 100F are not stretched. After that, the resistance, pretty much, levels off. This is seen more clearly in Table 3 and chart 420: the first 10% of stretch increases the resistance, averagely, by 14%. Further stretching the stripe by another 10%, with respect to the previous stretched state, the resistance increases, averagely, by just another 5.5%. After that, the resistance, pretty much, levels off at about 1%.

It should be noted that flat conductive stripe 100F is also more convenient logistically, than rounded conductive stripe 100R. For example, when using rolls of conductive stripes, the rolled up rounded conductive stripe 100R is much thicker and bulkier than the rolled up flat conductive stripe 100F.

The performance of conductive stripes 100 is also dependent on the behavior characteristics of the garment, having variable regional elasticity, and with which garment the conductive stripes 100 are coupled to operate. For example, a knitted garment is elastic by nature and therefore, the conductive stripes 100 have to adapt to the local elasticity of the garment. In normal operation, a conductive stripe 100 is typically stretched, with no limitations, by up to 25% with respect to length at the rest state of the conductive stripe 100. It should be noted that the elasticity of conductive stripes 100 should not limit the local elasticity of the garment.

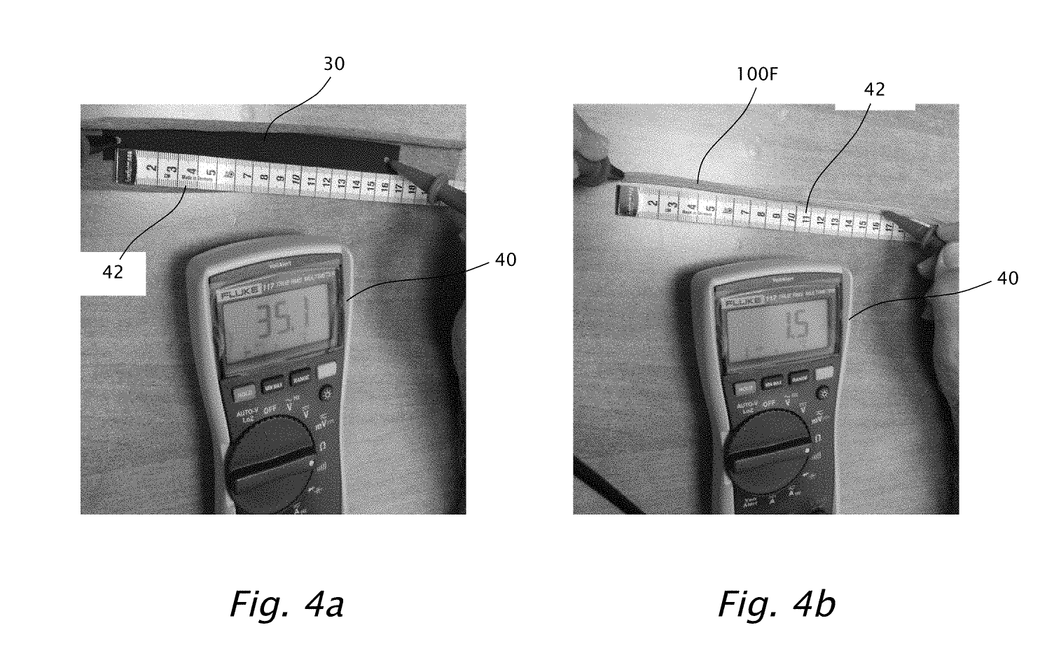

It should be noted that flat conductive stripe 100F was also compared to a prior art conductive stripe 30 that is made of elastic fabric having a mesh structure (as in Jeong), fabricated by XSTATIC. FIG. 4a depicts an example segment of a flat conductive stripe 100F having a length of 16 cm (as measured by ruler 42). FIG. 4b depicts an example prior art conductive stripe 30 having a compatible length of 16 cm. As shown, the electric resistance of the prior art conductive stripe 30 is 35 Ohms (as measured by device 40, depicted in FIG. 4a), while the electric resistance of the flat conductive stripe 100F of the present invention is only 1.5 Ohms. Hence the ability of the prior art conductive stripe 30 to transmit physiological signal sensed by a textile electrode is substantially poorer than that of flat conductive stripe 100F, while the sensitivity to motion artifacts, such as arms movements, is substantially higher. It is further noted that textile electrodes made from the same prior art fabric having a mesh structure, is by far inferior in the ability to a physiological signal, compared to the textile electrodes used in garment of the present invention. Therefore, a mesh structure based garment (as in Jeong) is not suited for measuring and transferring high quality physiological signals such as 12-lead clinical level ECG.

It should be noted that conductive stripes 100 can be made by knitting, weaving, braiding, or any other textile method which can combine both conductivity and elasticity. The good conductivity of conductive stripes 100 should prevail when using any type of basic fabric yarns to make the smart garment (such as synthetic yarns, metallic yarns, etc.).

Conductive stripes 100 is insulated to thereby prevent electrical shorting, while wearing and moving, for example, to prevent conductive stripes 100 from being electrically shortened by the user's skin, by neighboring conductive stripes 100 or neighboring textile electrode 50.

The insulation can be done by knitting, weaving, braiding, and covering, using any non-conductive textile material, natural or synthetic yarns.

The insulation should not reduce the conductivity and the elasticity properties of conductive stripes 100.

Conductive stripes 100 are positioned in a preconfigured configuration along the shirt to facilitate the stripes to stretch while wearing.

In one embodiment of the present invention, the insulation of conductive stripes 100 is done after the braiding process, using Spandex yarn covered with Nylon yarn.

In one embodiment of the present invention, conductive stripes 100 are made of braided conductive yarns (for example, with no limitations, conductive yarns that are manufactured by XSTATIC) together with spandex yarns, in order to reach the right level of elasticity. However, conductive stripes 100 may be made using any other conductive materials such as stainless steel yarns, cooper yarns and any other combination of conductive yarns), provided that the of conductive stripes 100 is similar to the local elasticity of the smart garment.

The basic yarns to knit the smart garment and the type of Spandex yarn used should be in line with the machine gauge and type of fabric requested.

The quantity of conductive yarn ends, elastic yarn ends, and the thickness (Den or Dtex) of the yarns in the braided stripe are determined by the level of conductivity and elasticity required for a particular smart garment.

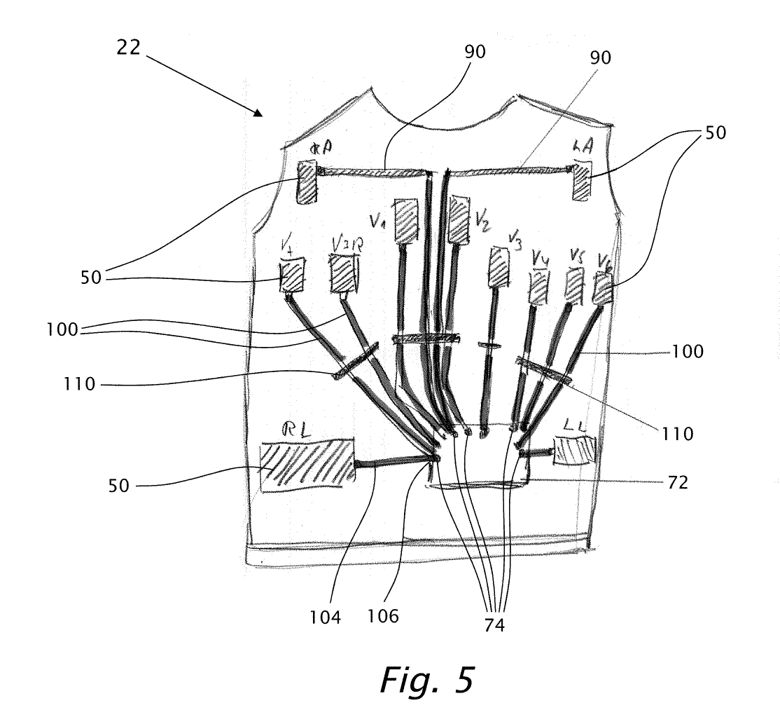

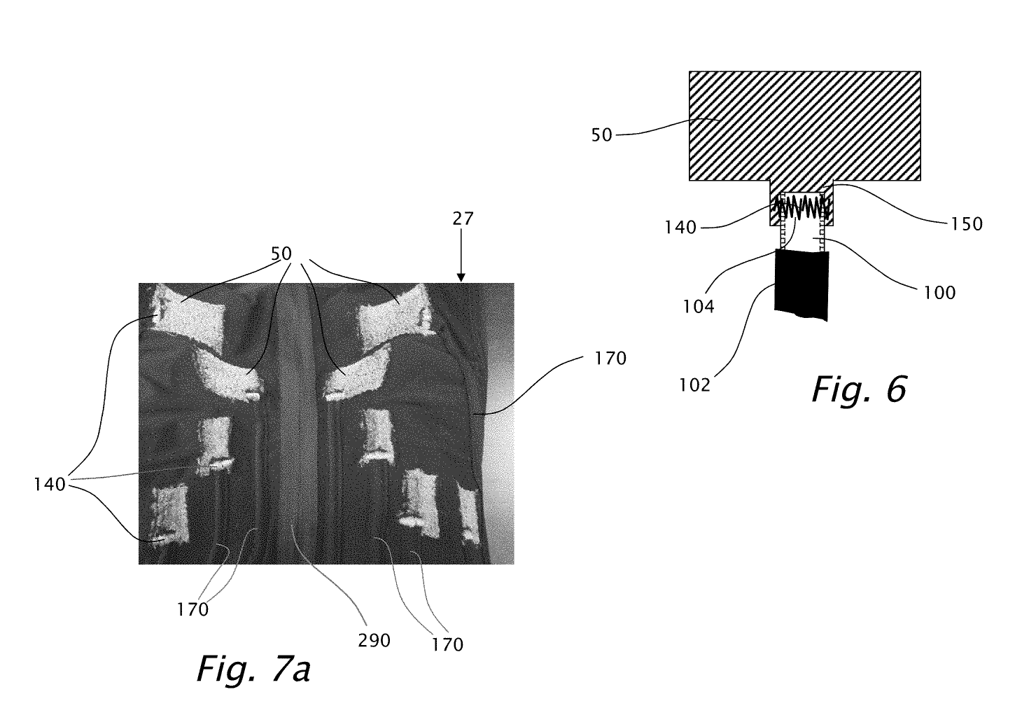

Reference is made to the drawings. FIG. 5 illustrates an example smart garment 22, having multiple textile electrodes 50 integrally knitted therein, wherein conductive stripes 100 are securely connected to respective textile electrodes 50, according to some embodiments of the present invention, facilitating the transfer of the sensed electrical signals from textile electrodes 50 to a target receiving unit such as a processing unit or a docking station 72. FIG. 6 illustrates an example method of securely connecting a conductive stripe 100 to a respective textile electrode 50, according to some embodiments of the present invention.

Smart garment 22, as shown by way of example only, with no limitations, as a knitted ECG monitoring shirt 22 having 13 knitted electrodes 50, integrally knitted therein (not all 13 electrodes shown) at preconfigured locations on the shirt 22. Each of the knitted electrodes 50 is adapted to detect an ECG signal that is transferred to the receiving unit.

In the example embodiment shown in FIG. 5, each elastic conductive stripe 100 of smart garment 22 is attached to elastic smart garment 22 at least at three locations: at a first endpoint 104 of conductive stripe 100 is securely and conductively attached to a respective textile electrode 50; at a second location, conductive stripe 100 is securely attached, for example adhered, is or passed through individual loops formed by a respective insulating stripe 110 that are secured to the garment, generally at middle of conductive stripe 100, and a second endpoint 106 of conductive stripe 100 is securely connected to the receiving unit at a respective location, being, in the example shown in FIG. 5, with no limitations, a respective snap 74 of docking station 72.

Elastic conductive stripes 100 are attached to smart garment 22 leaving some free segments hanging loosely between secured points to allow the garment fabric to stretch during wear without pulling the respective textile electrode 50 or minimizing the pulling force applied to the respective textile electrode 50. The elasticity of conductive stripe 100 also contributes to the minimization of the pulling force applied to the respective textile electrode 50.

The mechanical attachment of elastic conductive stripe 100 to textile electrode 50 must ensure the smooth and efficient transfer of the clinical level ECG signal from the textile electrode 50 to the respective conductive stripe 100. For example, as shown in FIG. 6, a first endpoint 104 of conductive stripe 100 is sewn (140) to the respective textile electrode 50 at lingula 150. Conductive stripe 100 may also be attached to the respective textile electrode 50 by lamination (adhesion) or by heat press. The attachment means does not reduce the conductivity of either the textile electrode 50 or the respective conductive stripe 100.

It should be noted that conductive stripes 100 may be attached to the shirt at the inner or the outer sides of smart garment 22.

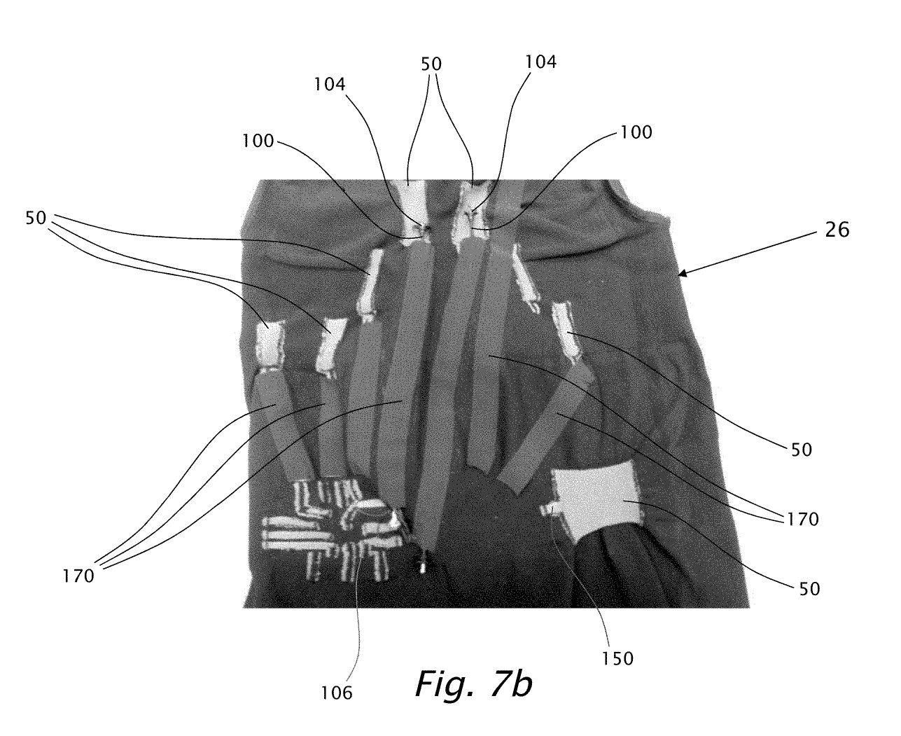

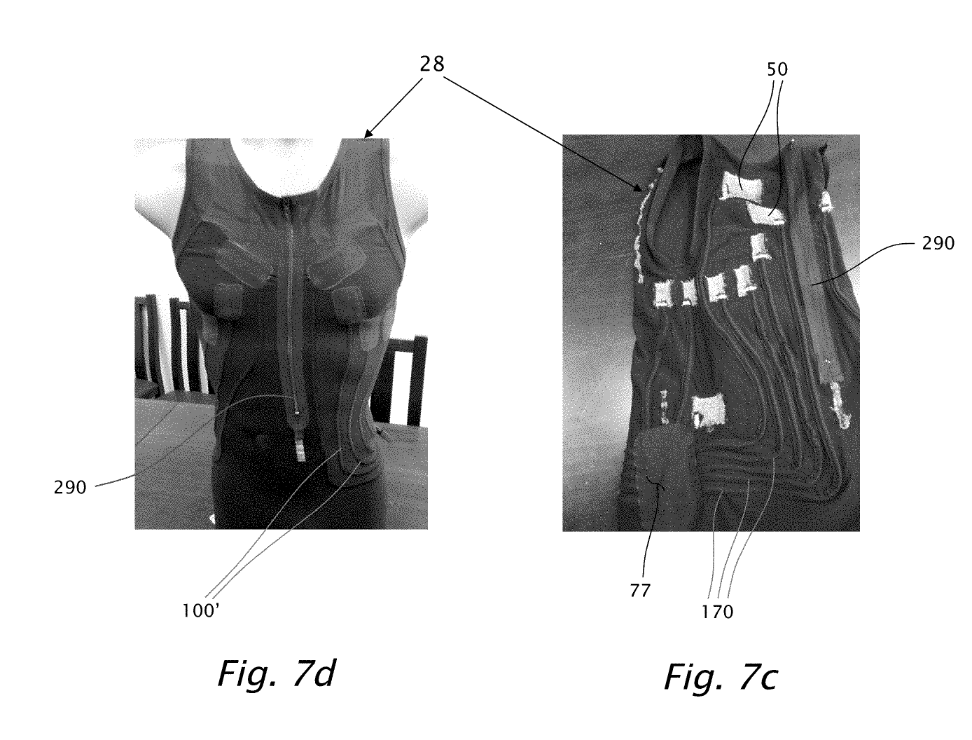

In some other embodiments of the present invention, each individual insulated conductive stripe 100 is inserted into a respective elastic sleeve which is securely attached to the fabric of the smart garment, for example by lamination. Reference is made to FIGS. 7a and 7b, depicting example methods of securely connecting a conductive stripe 100 to a respective textile electrode 50, according to other embodiments. FIG. 7b, illustrates example smart garments 26 and 27 (which garment 27 includes a zipper 290) showing the skin side of the garments, having multiple textile electrodes 50 connected to conductive stripes 100, wherein insulating sleeves 170 are used to insulate conductive stripes 100 from being electrically shortened by an adjacent conductive stripe and/or the user's skin.

All are inserted into respective sleeves 170, wherein a first endpoint 104 of the elastic conductive stripe 100 is securely connected, for example by sewing, to a respective textile electrode 50 and the other endpoint 106 of conductive stripe 100 is securely connected to a receiving unit, such as a docking station 72. Insulating sleeve 170 also keeps the accommodated elastic conductive stripe 100 hanging loosely between the two secured endpoints 104 and 106 of conductive stripe 100, to allow the garment fabric to stretch during wear without pulling the respective textile electrode 50, or minimizing the pulling force applied to the respective textile electrode 50. Insulating sleeve 170 serve as motion restricting means for the accommodated elastic conductive stripe 100, preventing sideways movements of the accommodated elastic conductive stripe 100 outside the boundaries of the respective insulating sleeve 170.

A laminated sleeve 170 of each of the conductive stripes 100, eliminates the need of insulating lining 160 to cover all conductive stripes 100. Sleeves 170 also keep each conductive stripe 100 in a preconfigured path along the fabric of the smart garment (such as garment 26 and 27).

FIGS. 7c and 7d depict another example garment 28, according to the methods shown in FIGS. 7a and 7b. FIG. 7c, illustrates the internal side (i.e., the skin side) of garment 28 (which garment 28 is a lady's garment that includes a zipper 290), having multiple textile electrodes 50 connected to respective conductive stripes 100, wherein insulating sleeves 170 are used to insulate conductive stripes 100 from being electrically shortened by an adjacent conductive stripe and/or the user's skin. FIG. 7d illustrates the external side of garment 28 showing the protrusions 100′ formed by the sewn-in (on the internal side of garment 28) conductive stripes 100. Sleeves 170 lead conductive stripes 100 towards a processing unit 70 (being on the external side of garment 28, not shown in FIG. 7d) that is accommodated inside a designated pocket 77.

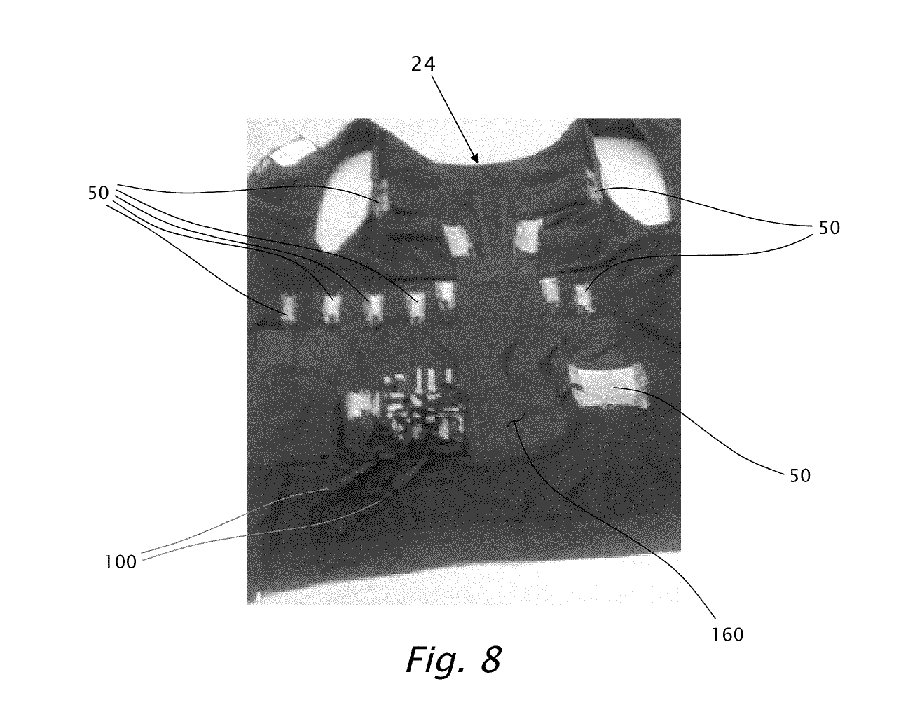

Reference is now also made to FIG. 8, showing the skin side of an example smart garment 24, having multiple textile electrodes 50 connected to conductive stripes 100, wherein the conductive stripes 100 are covered by a lining 160 that is used to insulate conductive stripes 100 from being electrically shortened by the user's skin, according to some embodiments of the present invention. Lining 160 facilitates each conductive stripe 100 to reach the designated conductive location 74 (see FIG. 5) at docking station 72.

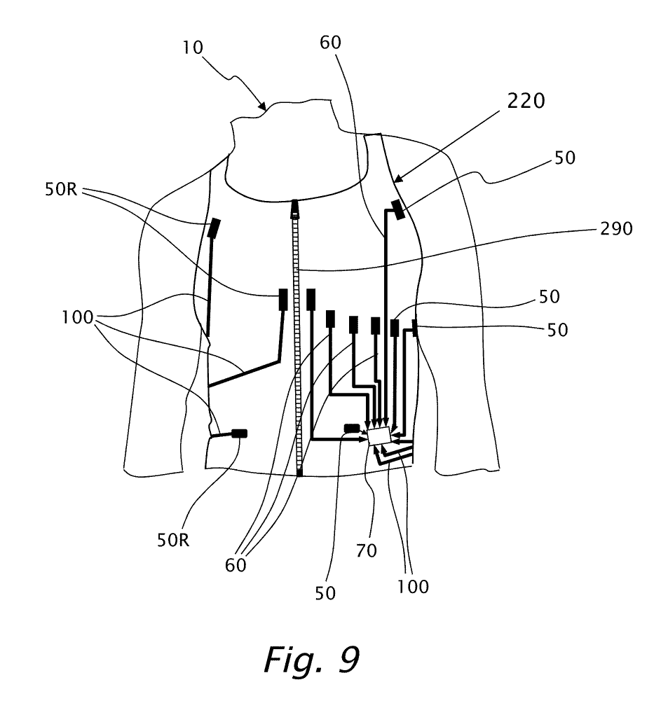

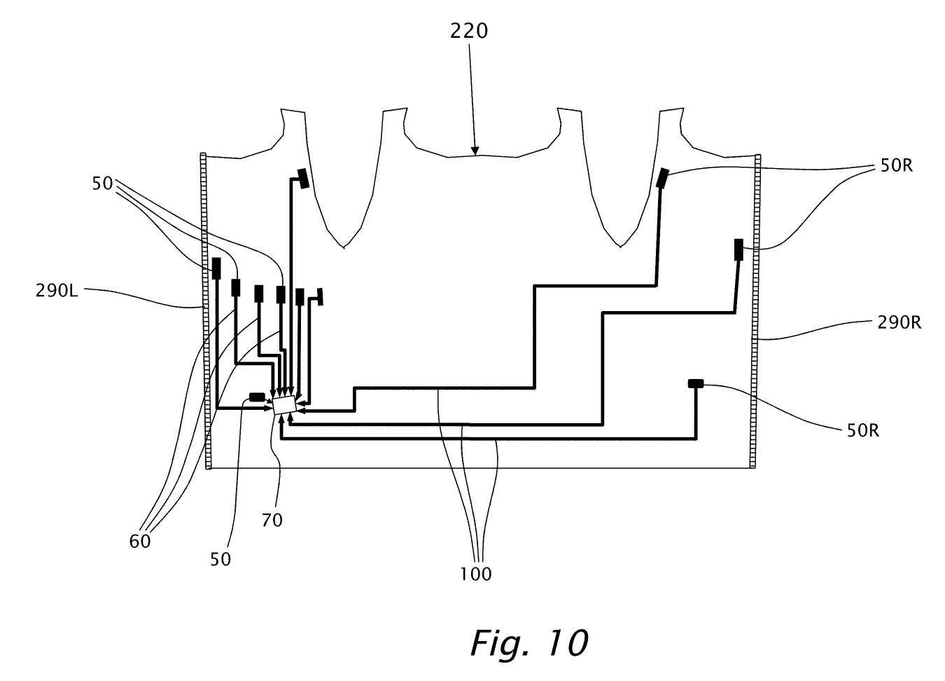

Reference in now made to FIG. 9, a schematic illustration of an example garment 220 having a tubular form, the garment being an undershirt having a zipper 290 in the front side, wherein textile electrodes 50 are knitted therein and are individually operatively connected to processing unit 70. However, some electrodes, such as textile electrodes 50R, may require crossing zipper 290. To overcome the problem conductive stripes 100 or line-traces (not shown) are knitted into or attached to smart garment 220 in a path that is traced around, via the back side of the garment, such as to bypass zipper 290. FIG. 10 is a schematic illustration of an example garment 220, as shown in FIG. 9, wherein zipper 290 is unzipped and the garment is in a spread, unfolded form.

The bypassing technique is also valid to any location of a generally vertical zipper, whereas conductive stripes 100 or knitted line-traces (not shown) are knitted into or attached to smart garment 220 in a path that is set to continuously pass through the continuous section of the garment between the 290L and 290R parts of zipper 290.

The invention being thus described in terms of embodiments and examples, it will be obvious that the same may be varied in many ways. Such variations are not to be regarded as a departure from the spirit and scope of the invention, and all such modifications as would be obvious to one skilled in the art are intended to be included within the scope of the claims.

What is claimed is:

1. An elastic smart garment, the garment comprising:
a) an elastic tubular form having a preconfigured variable elasticity, a skin side and an external side, wherein said external side faces away from the user's skin;
b) at least one conductive textile electrode for sensing an electrical vital signal, integrally manufactured with said tubular form; and
c) at least one elastic conductive stripe, having a first end and a second end,
wherein said first end of each said at least one conductive stripe is securely and conductively attached to a respective said conductive textile electrode, and wherein said second end of said at least one conductive stripe is operatively connected to a processor, facilitating said sensed vital signal to be communicated from said least one conductive textile electrode to said processor;
wherein said at least one conductive stripe is insulated by insulation means; and
wherein said at least one conductive stripe is at least partially detached from the garment, and wherein said elasticity of each said at least one conductive stripe, are configured to prevent or minimize the forming of a pulling force from being applied by said at least one conductive stripe to said respective conductive textile electrode, when the garment is stretched.

2. The garment of claim 1, wherein said elastic tubular form is a knitted tubular form.

3. The garment of claim 1, wherein said at least one conductive stripe is a flat conductive stripe or a rounded conductive stripe.

4. The garment of claim 1, wherein when said at least one conductive stripe is stretched in length by up to 15%, the electric resistance increases by less than 25%, with respect to the non-stretched, rest state of said at least one conductive stripe.

5. The garment of claim 4, wherein when said at least one conductive stripe is further stretched in length beyond the 15% and up to 30% of the rest state sate length, the electric resistance increases by less than 10%, with respect to the non-stretched, rest state of said at least one conductive stripe.

6. The garment of claim 5, wherein when said at least one conductive stripe is further stretched in length beyond the 30% of the rest state sate length, the electric resistance increases by less than 5%, with respect to the non-stretched, rest state of said at least one conductive stripe.

7. The garment of claim 1, wherein said electrical vital signal is a clinical-level ECG signal.

8. The garment of claim 1, wherein said at least one conductive stripe is composed of a plurality of yarns.

9. The garment of claim 1, wherein at least a portion of said yarns are braided.

10. The garment of claim 1, wherein at least a portion of said plurality of yarns are braided.

11. The garment of claim 1, wherein sideways movements of said at least one conductive stripe is restricted by a motion restricting means, wherein said motion restricting means is securely attached to the garment, and wherein said at least one conductive stripe is free to move within the restricted space as provided by said motion restricting means.

12. The garment of claim 11, wherein said motion restricting means is selected from the group of motion restricting means group consisting of a sleeve, sewn-in yarns that are sewn over said at least one conductive stripe, and a combination thereof.

13. The garment of claim 11, wherein said motion restricting means is securely attached to said skin side of the garment.

14. The garment of claim 1, wherein said insulation means is selected from the group consisting of at least one insulating adhered stripe, at least one sleeve, non-conductive coating and non-conductive textile material that is knitted, weaved, braided or covered on the respective at least one conductive stripe.

15. The garment of claim 1, wherein said insulation means is designed not to reduce the conductivity of the respective said at least one conductive stripe.

16. The garment of claim 1, wherein said insulation means is designed not to reduce the local elasticity of the respective said at least one conductive stripe.

17. The garment of claim 1, wherein said at least one conductive stripe is at least partially loose inside said insulation means.

18. The garment of claim 1, wherein said by insulation means is configured to prevent said at least one conductive stripe conductive stripe from being electrically shortened by any one of the user's skin, a neighboring conductive stripe or a neighboring textile electrode.

19. The garment of claim 8, wherein said yarns are selected from the group of yarns consisting of synthetic yarns, metallic yarns, and a combination thereof.

20. The garment of claim 1, wherein said second end of said at least one conductive stripe is securely attached to a connector or a docking station that is operatively connected to a processor.
